(19) United States
(12) Patent Application Publication
(10) Pub. No.: US 2011/0059024 A1
(43) Pub. Date: Mar. 10, 2011

(54) LIPOSOME COMPOSITION, AND DIAGNOSTIC CONTRAST AGENT, THERAPEUTIC ENHANCER, AND PHARMACEUTICAL COMPOSITION USING THE SAME

(75) Inventors: Hiroyuki HIRAI, Ashigarakami-gun (JP); Katsuro TACHIBANA, Fukuoka-shi (JP)

(73) Assignees: FUJIFILM CORPORATION, Tokyo (JP); FUKUOKA UNIVERSITY, Fukuoka-shi (JP)

(21) Appl. No.: 12/877,506

(22) Filed: Sep. 8, 2010

(30) Foreign Application Priority Data
Sep. 8, 2009 (JP) 2009-207270

(51) Int. Cl. A61K 49/22; A61K 9/127; A61P 35/00

(52) U.S. Cl. 424/9.51; 424/450

(57) ABSTRACT

To provide a liposome composition, which contains at least one liposome, gas entrapped in the liposome, and at least one fullerene encapsulated in or adsorbed on the liposome.

### BACKGROUND OF THE INVENTION

1. Field of the Invention

The present invention relates to a liposome composition containing at least one liposome, which entraps gas therein and encapsulates or adsorbs at least one fullerene therein or thereon, as well as relating to a diagnostic contrast agent, therapeutic enhancer, and pharmaceutical composition, all using such liposome composition.

2. Description of the Related Art

Use of fullerenes as a photosensitizer in a treatment for cancer or the like has been studied in recent year. This is an attempt to use oxidizability of various active oxygen, such as hydroxyl radicals, super oxide anions, singlet oxygen, generated by applying visible light or ultrasonic wave to a photosensitizer such as fullerenes. The ultrasonic radiation has a characteristic that it has a large permeation (affecting) distance in a water phase compared to the light radiation, and thus an application thereof to illness caused in the deep part of a body where light cannot reach has been expected (see, for example, J. W. Arbogast, A. P. Darmanyan, C. S. Foote, et al., J. Phys. Chem. (1991), 95, 11-12, Masatoshi Yamada, Yasuhiko Tabata, Medical and Biological Engineering (2005), 43, 238-246, and Japanese Patent Application Laid-Open (JP-A) No. 2002-241307).

The ultrasonic therapy for cancer or the like includes those using heat generated due to ultrasonic absorption by biotissues, those using mechanical functions of ultrasonic vibration, and a sonochemistry therapy in which a chemical reaction of a compound administered in a living body is induced by using a cavitation effect initiated by ultrasonic waves. There are various reports such that an application of ultrasonic waves to cancer cells leads apoptosis to thereby inhibit a growth of the cancer cells (see, for example, Q. Liu, X. Wang, P. Wang, et al., Ultrasonics (2006), 45, 56-60, H. Honda, Q. L. Zhao, T. Kondo, “Ultrasound” in Med. & Biol. 28 (2002) 673-682, JP-A No. 11-92360).

In the case where fullerenes are applied to living bodies as a medical material, as a surface of each fullerene is hydrophobic, they cannot be dispersed in a water medium as they are. There are various attempts to make fullerenes hydrophilic, such as a method of modifying a surface of a fullerene with a water-soluble polymer (see, for example, JP-A Nos. 2001-348214, 2006-69812, 2007-176899, and 2008-255107).

However, such fullerenes still have insufficient dispersion stability under neutral or approximately neutral physiological conditions, and thus they may cause aggregations. For this reason, it is difficult to secure sufficient fluidity in blood. Therefore, it is current situation that a fullerene dispersion liquid cannot be directly administered in a blood vessel as an injection.

Meanwhile, a liposome has been attempted to use for carrying particles into cells. The liposome is a vesicle formed of lipids that are also constitutional substances of a biological membrane, and has excellent compatibility to living bodies. In addition, it is possible to encapsulate various medicines in the vesicle. Therefore, the liposome has been widely used as a carrier for medicines. Moreover, since specificity to a cell or tissue can be provided to the liposome by changing the polarity, particle diameter or used lipid substances of the liposome, or bonding a specific ligand (e.g. an antigen, antibody, and sugar), the liposome has been attracted great attention as a drug carrier capable of targeting, and has been clinically applied as a carrier of a chemotherapeutic agent having a strong side effect, such as an anticancer agent (see, for example, A. Ikeda, Y. Doi, K. Nishiguchi, et al., Org. Biomol. Chem. (2007), 5, 1158-1160, and JP-A Nos. 05-58879, 2000-319165, and 2006-273740).

However, it is expected that a therapeutic effect obtainable by ultrasonic radiation reduces as particles are encapsulated in the liposome.

Recently, an ultrasonic contrast agent (SONAZOID, manufactured by Daiichi Sankyo Company, Limited) in which perflubutane (i.e. inert gas) is encapsulated in a liposome has been put on the market, but therapeutic use thereof has not been approved yet.

Moreover, it has been proposed a method in which a gas precursor which will be activated depending on a temperature is encapsulated in a liposome, and image diagnoses or heat treatments are carried out by using an increase of the temperature due to ultrasonic radiation to such liposome (see, for example, U.S. Pat. No. 7,078,015).

Although this method is simple and easy, the method has a dangerous possibility such that rapidly induced heat may damage the entire tissue.

Accordingly, it is the current situation that there is a strong demand for the immediate development of a liposome composition, which is excellent is dispersion stability under the approximately neutral physiological conditions, and is applicable for a diagnostic contrast agent, a therapeutic enhancer, and a pharmaceutical composition.

### BRIEF SUMMARY OF THE INVENTION

The present invention aims at solving various problems in the art and achieving the following object. Namely, an object of the present invention is to provide a liposome composition, which is excellent is dispersion stability under neutral or approximately neutral physiological conditions, and is applicable for a diagnostic contrast agent, a therapeutic enhancer, and a pharmaceutical composition, as well as providing a diagnostic contrast agent, a therapeutic enhancer, and a pharmaceutical composition, all using such liposome composition.

As a result of diligent studies and researches conducted by the present inventors, they have reached the following insight. That is, a liposome composition which contains at least one liposome entrapping gas therein, and encapsulating or adsorbing at least one fullerene therein or thereon, is excellent in dispersion stability under neutral or approximately neutral physiological conditions, and is applicable for a diagnostic contrast agent, a therapeutic enhancer, and a pharmaceutical composition.

The present invention is based upon the insight of the present inventors, and means for solving the aforementioned problems are as follows.

<1> A liposome composition, containing:

at least one liposome;

gas entrapped in the liposome; and

at least one fullerene encapsulated in or adsorbed on the liposome.

<2> The liposome composition according to <1>, wherein the liposome composition has a volume average dispersed-particle diameter of 20 nm to 20 μm.
<3> The liposome composition according to any of <1> or <2>, wherein the gas is at least one selected from the group consisting of oxygen, nitrogen, carbon dioxide, xenon, krypton, argon, hydrofluorocarbons, and perfluorocarbons.
<4> The liposome composition according to any one of <1> to <3>, wherein the fullerene is at least one selected from the group consisting of C₆₀, C₇₀, and derivatives thereof.
<5> The liposome composition according to <4>, wherein the derivative is at least one selected from the group consisting of C₆₀ to which at least one group selected from —OH and —COOH is added, and C₇₀ to which at least one group selected from —OH and —COOH is added.
<6> The liposome composition according to any one of <1> to <5>, further containing a receptor bonded to or contained in the liposome, wherein the receptor is capable of specifically recognizing a certain tissue.
<7> The liposome composition according to any one of <1> to <6>, wherein the liposome composition is ultrasonic sensitive.
<8> The liposome composition according to any one of <1> to <7>, wherein the liposome composition is used for a medical use.
<9> A diagnostic contrast agent containing the liposome composition as defined in any one of <1> to <8>.
<10> A therapeutic enhancer containing the liposome composition as defined in any one of <1> to <8>.
<11> A pharmaceutical composition containing the liposome composition as defined in any one of <1> to <8>.
<12> A diagnose method, containing administering the diagnostic contrast agent as defined in <9> to a body.
<13> A method for enhancing a therapy, containing administering the therapeutic enhancer as defined in <10> to a body.
<14> A therapeutic method, containing administering the pharmaceutical composition as defined in <11> to a body.

The present invention contributes to solve various problems in the art, and provides a liposome composition, which is excellent is dispersion stability under neutral or approximately neutral physiological conditions, and is applicable for a diagnostic contrast agent, a therapeutic enhancer, and a pharmaceutical composition, as well as providing a diagnostic contrast agent, a therapeutic enhancer, and a pharmaceutical composition, all using such liposome composition.

### BRIEF DESCRIPTION OF THE DRAWINGS

FIG. 1 is a schematic diagram showing, as one embodiment of the present invention, a liposome composition containing a liposome which entraps gas therein, and adsorbs fullerenes. In FIG. 1, “11” is a liposome composition, “17” is a liposome, “12” is a hydrophilic part, “13” is a hydrophobic part, “14” is a fullerene (a surface of which may be modified with a hydrophilic compound), “15” is gas (which may be covered with a lipid), and “16” is a receptor.

FIG. 2 is a schematic diagram showing, as another embodiment of the present invention, showing a liposome composition containing a liposome which contains gas therein, and encapsulates fullerenes. In FIG. 2, “21” is a liposome composition, “27” is a liposome, “22” is a hydrophilic part, “23” is a hydrophobic part, “24” is a fullerene (a surface of which may be modified with a hydrophilic compound), “25” is gas (which may be covered with a lipid), and “26” is a receptor.

FIG. 3A is a schematic diagram showing one example of the positioning of the liposome, fullerenes, and gas in the liposome composition of the present invention. In FIG. 3A, “31” is a liposome composition, “36” is a liposome, “32” is a fullerene, “33” is an aqueous solution, and “34” is gas.

FIG. 3B is a schematic diagram showing another example of the positioning of the liposome, fullerenes, and gas in the liposome composition of the present invention. In FIG. 3B, “31” is a liposome composition, “36” is a liposome, “32” is a fullerene, and “34” is gas.

FIG. 3C is a schematic diagram showing another example of the positioning of the liposome, fullerenes, and gas in the liposome composition of the present invention. In FIG. 3C, “31” is a liposome composition, “36” is a liposome, “32” is a fullerene, and “34” is gas.

FIG. 3D is a schematic diagram showing another example of the positioning of the liposome, fullerenes, and gas in the liposome composition of the present invention. In FIG. 3D, “31” is a liposome composition, “36” is a liposome, “35” is a fullerene (an aggregate of fullerenes), and “34” is gas.

FIG. 3E is a schematic diagram showing another example of the positioning of the liposome, fullerenes, and gas in the liposome composition of the present invention. In FIG. 3E, “31” is a liposome composition, “36” is a liposome, “32” is a fullerene, “34” is gas, and “35” is a fullerene (an aggregate of fullerenes).

### DETAILED DESCRIPTION OF THE INVENTION

### Liposome Composition

The liposome composition of the present invention contains at least one liposome, gas entrapped in the liposome, and at least one fullerene encapsulated in or adsorbed onto the liposome, and may further contain other substances, if necessary.

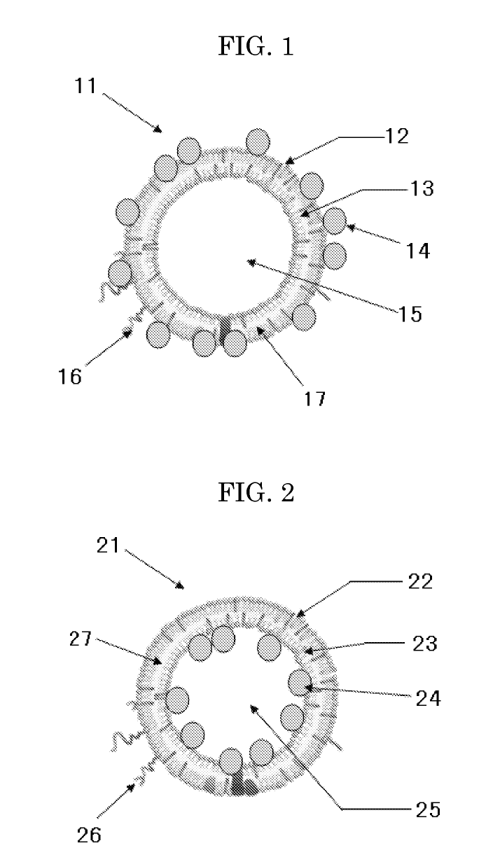

Embodiments of the liposome composition will be explained with reference to FIGS. 1 to 3E.

FIG. 1 is a schematic diagram showing, as one embodiment of the present invention, a liposome composition containing a liposome which contains gas in the liposome and adsorbs fullerenes on the liposome. In FIG. 1, the gas is contained in the space present in the center part of the liposome, and the fullerene is adsorbed by the hydrophilic part of the liposome. In addition, a receptor is bonded to the liposome.

FIG. 2 is a schematic diagram showing, as another embodiment of the present invention, a liposome composition containing a liposome which entraps gas and encapsulates fullerenes in the liposome. In FIG. 2, the gas is contained in the space present in the center part of the liposome, and the fullerene is encapsulated in the liposome. In addition, a receptor is bonded to the liposome.

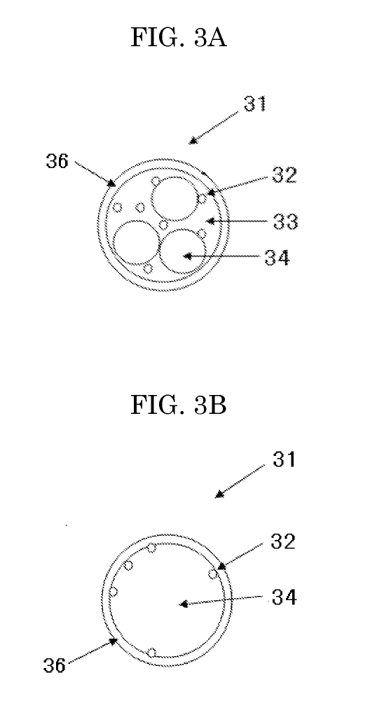

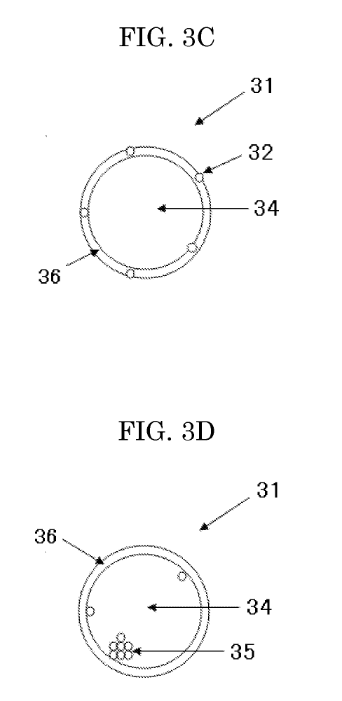

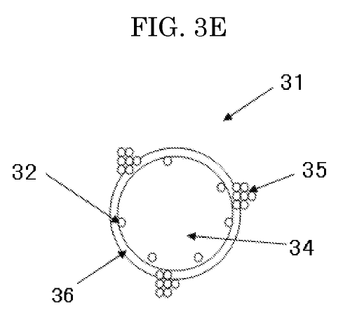

FIGS. 3A to 3E are schematic diagrams showing examples of the positioning of the liposome, fullerenes, and gas in the liposome composition of the present invention. In FIG. 3A, the gas coated with a lipid and the fullerenes are present in the center part of the liposome, and the rest of the center part is filled with an aqueous solution. In FIG. 3B, the gas is present in the space at the center part of the liposome, and the fullerenes are encapsulated in the liposome. In FIG. 3C, the gas is present in the space at the center part of the liposome, and the fullerenes are adsorbed on the hydrophilic part. In FIG. 3D, the gas is present in the space at the center part of the liposome, and fullerenes are encapsulated in the liposome, where part of the fullerenes are present as an aggregate. In FIG. 3E, the gas is present in the space at the center part of the liposome, and fullerenes are encapsulated in and adsorbed on the liposome, where part of the fullerenes are present as aggregates.

The gas may be covered with a lipid. Moreover, the gas may be present in the hydrophobic part of the liposome.

The surface of the fullerene may be coated with a hydrophilic compound, and such fullerene may be encapsulated and present in the space at the center part of the liposome.

The liposome composition of the present invention may be formed of a single layer membrane or multilayer membrane containing two or more layers. Moreover, the lipid covering the gas may be made out of the same or different lipid for forming the liposome.

In the liposome composition of the present invention, the liposome entrapping the gas therein and the fullerene(s) are each present to have a distance with which an interaction between the liposome and the fullerene(s) can be initiated by ultrasonic radiation.

The interaction is for example to exhibit a synergistic effect by superimposing the regain where the gas exhibits a cavitation effect by adsorbing ultrasonic waves, and the region where active oxygen generated by the fullerene(s) is present.

### <Volume Average Dispersed-Particle Diameter>

The volume average dispersed-particle diameter of the liposome composition, which containing at least one liposome entrapping the gas therein and encapsulating or adsorbing at least one fullerene, is suitably selected depending on the intended purpose without any restriction. The volume average dispersed-particle diameter thereof is preferably 20 nm to 20 μm, and more preferably 50 nm to 10 μm. When the volume average dispersed-particle diameter thereof is less than 20 nm, it is difficult to synthesize a liposome itself, and is also difficult to stably contain the gas or fullerene(s) in the liposome. When the volume average dispersed-particle diameter thereof is more than 20 μm, vascular occlusion or hematogenous disorder may occur in capillary vessels or a part of a vessel where a blood flow is slow, and the liposome composition may not readily reach an affected part, such as cancer cells. When the volume average dispersed-particle diameter thereof is in the aforementioned preferable range, on the other hand, sufficient dispersion stability and fluidity can be attained in a solution such as a blood stream so that such liposome can be used for medical purpose such as diagnoses and treatments. Therefore, the liposome with such volume average dispersed-particle diameter is advantageous.

In the case where the liposome composition is used as a diagnostic contrast agent, the volume average dispersed-particle diameter of the liposome composition is suitably selected depending on the intended purpose without any restriction, but it is preferably 100 nm to 20 μm, more preferably 1 μm to 10 μm. When the volume average dispersed-particle diameter thereof in the more preferable range, it is advantageous because the liposome composition tends to provide a clear contrast in a resulting image.

In the case where the liposome composition is used as a therapeutic enhancer, the volume average dispersed-particle diameter thereof is suitably selected depending on the intended purpose without any restriction. For example, in case of a cancer treatment, it is preferably 50 nm to 500 nm, more preferably 60 nm to 300 nm. When the volume average dispersed-particle diameter thereof is in the more preferable range, it is possible to preferentially accumulate such liposome composition onto cancer tissues due to an enhanced permeation and retention effect (EPR effect), and thus it is effective in the enhancement of the cancer treatment.

The volume average dispersed-particle diameter of the liposome composition can be measured by dynamic light scattering. For example, it can be measured by means of a microtrack UPA-UT151 particle size distribution analyzer (manufactured by Nikkiso Co., Ltd.).

The reason is not clear why the effect in diagnoses and treatments increases when the gas and the fullerene(s) are used in combination, compared to the case where either of them is used independently. However, it is probably because the fullerene(s) moves more intensely with assistance of buoyancy of the gas, for example by ultrasonic radiation, in the closed space like the liposome. Compared to the case of a liposome itself or a bubble liposome containing gas, it is assumed that the liposome composition of the present invention increases ultrasonic therapeutic effect by involving different action mechanisms. The positioning of the liposome, fullerene(s) and gas is shown in FIGS. 3A to 3E. By using the aforementioned technique, the ultrasonic sensitivity of the liposome composition can be enhanced.

### <Ultrasonic Sensitivity>

The liposome composition is preferably ultrasonic sensitive, as it will provide the liposome composition with a therapeutic effect or diagnostic effect for cancer or the like.

Being ultrasonic sensitive means that the liposome composition is heated, receives mechanical vibrations, or exhibits a cavitation effect by ultrasonic radiation.

By applying ultrasonic waves to the liposome composition containing at least one liposome in which the gas and the fullerene(s) are both present, the obtainable effect (e.g. a bactericidal effect in dental treatments, and an effect of killing or damaging cancer cells) significantly improves.

### <Gas>

The gas is suitably selected depending on the intended purpose without any restriction, provided that it can be entrapped in the liposome. The gas is preferably selected from those being present as a vapor under physiological conditions.

“Physiological conditions” means that it is in phosphate buffered saline (composition: 137 mM-NaCl, 9.0 mM-Na₂HPO₄, 2.9 mM-NaH₂PO₄) having a pH value of 7.2 to 7.4, at 25° C., and 1 atm.

Examples of the preferable gas include oxygen, nitrogen, carbon dioxide, xenon, krypton, argon, hydrofluorocarbons, and perfluorocarbons. These may be used independently, or in combination.

Among them, xenon, krypton, argon, hydrofluorocarbons, and perfluorocarbons are advantageously used. This is because these are insoluble in water, and molecular size and density thereof are large so that these can be stably contained within the liposome, which leads high sensitivity for diagnoses, and high therapeutic effect.

Examples of the hydrofluorocarbons include 1,1,1,2,2-pentafluoroethane, 1,1,2,2-tetrafluoroethane, 1,1,1-trifluoroethane, 1,1-difluoroethane, 1,1,1,2,3,3,3-heptafluoropropane, 1,1,1,3,3,3-hexafluoropropane, 1,1,2,2,3-pentafluoropropane, and 1,1,1,2,3,4,4,5,5,5-decafluoropentane.

Examples of the perfluorocarbons include those known as ultrasonic contrast agents, such as perfluoroethane, perfluoropropane, perfluorobutane, perfluorocyclobutane, perfluoropentane, and hexafluoro-1,3-butadiene.

The amount of the gas contained in the liposome composition is suitably selected depending on the intended purpose without any restriction, provided that it is equal to or smaller than the volume of the void(s) of the liposome composition. The amount of the gas is preferably 10% to 100%, more preferably 20% to 95%, and even more preferably 25% to 90% relative to the volume of the void(s) of the liposome composition. When the amount of the gas is less than 10%, the obtainable therapeutic effect is small. When the amount thereof is more than 100%, the condition of the liposome composition becomes unstable. On the other hand, when the amount of the gas contained in the liposome composition is in the aforementioned even more preferable range, it is advantageous, as sensitivity for diagnoses increases, and a significant effect of enhancing treatments can be attained.

The amount of the gas contained in the liposome composition can be assumed, for example, by obtaining an amount of the gas by gas chromatography or the like, and comparing the obtained value with the size of the liposome composition measured by an optical microscope or electron microscope.

Moreover, an amount of the gas contained in a dispersion liquid, in which the liposome composition of the present invention is dispersed, is suitably selected depending on the intended purpose without any restriction. The amount thereof is preferably 0.1 μL to 100 μL per 1 mL. When the amount of the gas in the dispersion liquid in which the liposome composition is dispersed is less than 0.1 μL, an effect of diagnoses and an effect of enhancing treatments are not obtained. In this case, moreover, a pharmacological agent cannot be administered in a uniform concentration because the liposome composition containing the fullerene(s) is precipitated in a storage container, which may cause a significant accident. When the amount of the gas is more than 100 μL, the dispersion liquid is unstable so that the liposome composition is floated in the container. Therefore, a pharmacological agent cannot be administered in a uniform concentration, which may cause a significant accident. On the other hand, when the amount of the gas contained in the dispersion liquid in which the liposome composition is dispersed is within the aforementioned preferable range, it is advantageous because the liposome composition is stably present so that sensitivity for diagnoses increases and a significant effect of enhancing treatments can be attained.

The gas may be covered with a lipid. Moreover, the gas may be present in the hydrophobic part of the liposome.

### <Fullerene>

The fullerene for use in the present invention means Cₙ(carbon) clusters on the whole.

The fullerene is suitably selected from fullerenes known in the art depending on the intended purpose without any restriction. Examples of the fullerene include: pure carbon materials such as C₆₀, C₇₀, C₇₆, C₇₈, and C₈₂; carbon clusters in which a metal (or metal oxide) is included; and fullerenes each of which is modified with a OH group(s) to enhance water-solubility thereof. These may be used independently, or in combination.

Among them, C₆₀, C₇₀, and derivatives thereof are preferable because they are readily available.

The aforementioned derivative is suitably selected depending on the intended purpose without any restriction. Examples thereof include C₆₀ to which at least one group selected from —OH and —COOH is added, and C₇₀ to which at least one group selected from —OH and —COOH is added.

The fullerene may be used in the aggregated state or non-aggregated state. When the fullerenes are not aggregated, the volume average particle diameter of the fullerenes is approximately 1 nm.

The volume average particle diameter of the aggregated fullerenes is suitably selected depending on the intended purpose without any restriction, but it is preferably 100 nm or less, more preferably 50 nm or less. When the volume average particle diameter of the aggregated fullerenes is more than 100 nm, it is difficult to encapsulate or adsorb the aggregated fullerenes in or on the liposome. On the other hand, when the volume average particle diameter of the aggregated fullerenes is within the aforementioned more preferable range, it is advantageous because the aggregated fullerenes are easily encapsulated in or adsorbed on the liposome so that ultrasonic treatments are enhanced by exhibiting synergistic effect with the gas.

A transmittance electron microscope (TEM) can be used for determination of the volume average particle diameter.

The volume average particle diameter means a diameter of a circle which is determined to have the same area to that of the image of the aggregated fullerenes taken by an electron microscopic photography.

Since the fullerene itself is generally water insoluble, it is difficult to administer the fullerene to living bodies. Therefore, it is preferred that the fullerene be treated to have hydrophilicity.

The method of the hydrophilication treatment is suitably selected from those known in the art depending on the intended purpose without any restriction. Examples thereof include the methods disclosed in JP-A Nos. 2001-348214, 2006-69812, 2007-176899, and 2008-255107.

The amount of the fullerenes relative to the total amount of lipids in the liposome is suitably selected depending on the intended purpose without any restriction, but it is preferably 0.1% to 10,000%, more preferably 1% to 2,000% based on a mass ratio {(fullerenes/the total lipids of the liposome)×100}. When the amount of the fullerene is less than 0.1%, the synergistic effect due to the combination of the gas and the fullerene may not be attained. When the amount thereof is more than 10,000%, it is difficult to stably encapsulate or adsorb the fullerene(s) in or on the liposome. On the other hand, when the amount thereof is in the aforementioned more preferable range, it is advantageous because the synergistic effect with the gas can be attained so that ultrasonic treatments are enhanced.

The fullerene(s) may be encapsulated in, or adsorbed on the liposome, or may be both.

Especially in the case where the liposome composition is used as a therapeutic enhancer or pharmaceutical composition, an embodiment in which the fullerene(s) is adsorbed on the outer side of the liposome is preferable for the following reason. For example, when by-products such as hydroxyl radicals and singlet oxygen are utilized, the generated hydroxyl radicals or singlet oxygen is shielded by the wall of the liposome so that the aforementioned active oxygen can easily and directly effect on an affected part by ultrasonic radiation. Accordingly, an effect of enhancing treatments can be attained.

In the case where a cavitation effect or mechanical function is expected, an embodiment in which the fullerene(s) is encapsulated in the liposome is preferable because the liposome is expected to break to open due to sonoporation to thereby attaining an effect of enhancing treatments.

Use of the fullerenes is advantageous over use of metal oxides such as iron oxide, as the fullerene has a small specific gravity so that it tends not to precipitate in a fluid.

### <Liposome>

The liposome for used in the liposome composition of the present invention, which includes the gas therein and encapsulates or adsorbs the fullerene(s) therein or thereon, is a closed vesicle containing a neutral lipid, and a negatively-charged lipid and/or a positively-charged compound. The lipid may be further bonded with a nonionic water-soluble polymer or protein.

The neutral lipid is a lipid having cations and anions in the equivalent numbers in a physiologic pH aqueous medium, namely an aqueous medium having a pH value of 6.5 to 7.5.

The neutral lipid is suitably selected depending on the intended purpose without any restriction. Examples thereof include: phosphatidic acid derivatives such as dipalmitoylphosphatidylcholine, and phosphatidylethanol amine; glycolipids such as digalactosyl glyceride, and galactosyl glyceride; sphingosine derivatives such as sphingomyelin; and sterols such as cholesterol, ergosterol, and lanosterol. These may be used independently or in combination.

Among them, the phosphatidic acid derivatives, glycolipids, and sterols are preferable, the phosphatidic acid derivatives and sterols are more preferable, and the phosphatidic acid derivatives are even more preferable.

Among the phosphatidic acid derivatives, di(C10-22 alkanoyl or alkenoyl) phosphatidylcholine derivatives are preferable, and dipalmitoylphosphatidylcholine, and distearoyl-sn-glycero-phosphatidylcholine are more preferable.

Examples of the aforementioned C10-22 alkanoyl or alkenoyl group include a decylyl group, an undecylyl group, a dodecylyl group, a tridecylyl group, a tetradecylyl group, a pentadecylyl group, a hexadecylyl group, a heptadecylyl group, an octadecylyl group, a nonadecylyl group, an icosyl group, a henicosyl group, a docosyl group, a decenyl group, a dodecenyl group, a tetradecenyl group, a hexadecenyl group, an octadecenyl group, an icocenyl group, and a dococenyl group.

The aforementioned “di(C10-22 alkanoyl or alkenoyl)” means that two hydroxyl groups contained in phosphatidylcholine are each esterification-bonded to a carboxylic acid of the C10-22 alkanoyl or alkenoyl group.

The sterols such as cholesterol themselves can be used as a constitutional component of the liposome, they ma be used, if necessary, added to other neutral lipids.

The negatively-charged lipid is a lipid having more cations than anions in a physiologic pH aqueous medium.

The negatively-charged lipid is suitably selected depending on the intended purpose without any restriction. Examples thereof include hydrogenated egg phosphatidylserine sodium salt; phosphatidylglycerols such as dipalmitoylphosphatidylglycerol; phosphatidylserines such as dipalmitoylphosphatidylserine; and phosphatidylinositols such as dipalmitoylphosphatidylinositol. These may be used independently or in combination.

Among them, phosphatidylglycerols are preferable, and dipalmitoylphosphatidylglycerol is more preferable.

The positively-charged compound is a compound having more anions than cations in a physiologic pH aqueous medium.

The positively-charged compound is suitably selected depending on the intended purpose without any restriction. Examples thereof include a positively-charged lipid, a cationic surfactant, and a cationic water-soluble polymer. These may be used independently or in combination.

The positively-charged lipid is suitably selected depending on the intended purpose without any restriction. Examples thereof include: chain hydrocarbon amines such as stearyl amine, and oleyl amine; amine derivatives of cholesterol such as 3-β-[N-(N′,N′-dimethylaminoethane)carbamoyl] cholesterol; N-α-trimethylammonioacetyl di(C10-20 alkyl or alkenyl)-D-glutamate chlorides such as N-α-trimethylammonioacetyldidodecyl-D-glutamate chloride; and N-[1-(2,3-di(C10-20 alkyl or alkenyl)oxy)propyl]-N,N,N-trimethylammonium chlorides such as N-[1-(2,3-dioleyloxy)propyl]-N,N,N-trimethylammonium chloride.

Examples of the alkyl group include a pentyl group, a hexyl group, an octyl group, a nonyl group, a decyl group, a dodecyl group, a tetradecyl group, a hexadecyl group, an octadecyl group, an icosyl group, a docosyl group, a tetracosyl group, a hexacosyl group, an octacosyl group, and a triacontasyl group. Among them, C5-30 alkyl groups are preferable, and C10-20 alkyl groups are more preferable.

Examples of C10-20 alkyl or alkenyl group include a decyl group, an undecyl group, a dodecyl group, a tridecyl group, a tetradecyl group, a pentadecyl group, a hexadecyl group, a heptadecyl group, an octadecyl, a nonadecyl group, an icosyl group, a decenyl group, a decynyl group, an undecynyl group, a dodecynyl group, and a tridecynyl group.

Among these positively-charged lipids, alkyl amine, N-α-trimethylammonioacetyl di(C10-20 alkyl or alkenyl)-D-glutamate chloride are preferable, and N-α-trimethylammonioacetyldidodecyl-D-glutamate chloride is more preferable.

The cationic surfactant is suitably selected cationic surfactants known in the art without any restriction. Examples thereof include cationic surfactants disclosed in M. J. ROSEN, (Tsubone, Sakamoto, trans.), Surfactants and Interfacial Phenomena (Fragrance Journal Ltd., 1995), pp. 16-20. The cationic surfactant may be used independently or in combination.

Among the cationic surfactants, long-chain alkyl amine and salts thereof, long-chain alkyl or aralkyl quaternary ammonium salt, polyoxyethylene adduct of long-chain alkyl amine or salts thereof, polyoxyethylene adduct of long-chain alkyl quaternary ammonium salt, and long-chain alkyl amine oxide are preferable, the long-chain alkyl amine or salts thereof, long-chain alkyl or alkenyl quaternary ammonium salt, and polyoxyethylene adduct of long-chain alkyl amine or salts thereof are more preferable, and the long-chain alkyl amine or salts thereof is even more preferable.

A highly concentrated cationic surfactant may destroy the liposome, but a cationic surfactant can be contained in the liposome as a component, if it is in a small amount (see Urbaneja et al., Biochem. J, vol. 270, pp. 305-308, 1990). Accordingly, by adding an amount of the cationic surfactant, which will not adversely affect the formation of the liposome, or which will not destroy the formed liposome, or adding the cationic surfactant in a dispersion liquid in which the previously formed liposome is dispersed to adsorb the cationic surfactant on the surface of the liposome, the cationic surfactant can be present as a component of the liposome to reduce the negatively-charged state of the polymer-modified liposome. This is preferable because the toxicity to living bodies can be reduced.

The cationic water-soluble polymer is suitably selected from cationic water-soluble polymers known in the art without any restriction. Examples thereof include cationic water-soluble polymers disclosed in G. Allen et al., edit., Comprehensive polymer science, (PergamonPress, 1989) vol. 6. The cationic water-soluble polymer may be used independently or in combination.

Among the aforementioned cationic water-soluble polymers, cationic water-soluble vinyl synthesized polymer, cationic water-soluble polyamino acid, cationic water-soluble synthesized polypeptide, cationic water-soluble natural polymer, and cationic water-soluble modified natural polymer are preferable, the cationic water-soluble vinyl synthesized polymer, cationic water-soluble polyamino acid, and cationic water-soluble synthesized polypeptide are more preferable, and the cationic water-soluble vinyl synthesized polymer is even more preferable.

The manner of the absorption of these cationic water-soluble polymers onto the liposome is different from the manner of absorption of a low-molecular weight compound thereto. The absorption of the polymer to a surface of a solid is stable, and irreversible (see G. Allen et al., edit., Comprehensive polymer science, (Pergamon Press, 1989), vol. 2, pp. 733-754. Accordingly, by adsorbing the cationic water-soluble polymer onto the negatively charged liposome, the negative charges of the liposome reduce. It is preferable because the toxicity to living bodies can be reduced.

In the present invention, each lipid may be bonded to a nonionic water-soluble polymer.

The nonionic water-soluble polymer is suitably selected depending on the intended purpose without any restriction, but preferable examples thereof include: nonionic polyether such as polyethylene glycol; nonionic monoalkoxy polyether such as monomethoxy polyethylene glycol, and monoethoxy polyethylene glycol; nonionic polyamino acid; and nonionic synthesized polypeptide.

The weight average molecular weight of the nonionic water-soluble polymer is suitably selected depending on the intended purpose without any restriction, but it is preferably 1,000 to 12,000, more preferably 1,000 to 5,000.

The diameter of the liposome (i.e. the liposome before including the gas therein) is suitably selected depending on the intended purpose without any restriction. Although the diameter thereof is different depending on how the size of the liposome is controlled, the volume average particle diameter of the liposome is preferably 10 nm to 500 nm, more preferably 20 nm to 200 nm, and even more preferably 20 nm to 100 nm.

Here, the volume average particle diameter means an average value of the particle diameters calculated from the average volume of a plurality of particles, and is calculated by means of a particle size analyzer in accordance with methods known in the art (e.g., R. R. C. New, edit., Liposomes: a practical approach (IRL Press, 1989), pp. 154-160).

### <Other Substances>

Other substances may be suitably selected depending on the intended purpose without any restriction, provided that they do not adversely affect the obtainable effect of the present invention. Examples thereof include a receptor.

### —Receptor—

It is preferable that the liposome composition of the present invention be bonded to or contain a receptor capable of specifically recognizing a certain tissue, because it is effective in diagnoses or treatments for tumors by ultrasonic waves, and it exhibits an effect of instructing killer cells.

The receptor is suitably selected depending on the intended purpose without any restriction. Examples thereof include various receptors that are accumulated specific to abnormal cells such as tumors. These may be used independently, or in combination.

Specific examples of the receptor include various monoclonal antibodies, various proteins, polypeptides, steroids, and immunity-related agents (e.g. immunocyte reactivation substances, activation substances).

The receptor is bonded to or contained in the liposome via a terminal amino group, hydroxyl group or carboxyl group of the aforementioned lipid, water-soluble polymer, or surfactant.

The receptor may cover the entire surface of the liposome, or part of the surface thereof.

### <Production Method>

The production method of the liposome composition containing at least one liposome which entraps the gas therein, and encapsulates or adsorb at least one fullerene therein or thereon, is suitably selected depending on the intended purpose without any restriction.

One embodiment of the production method thereof will be shown below.

1. A fullerene dispersion liquid is prepared.
2. Liposomes are formed by combining two or more lipids. Here, the softness of the liposome membrane may be changed (using the deference in the phase-transition points), or domains each having different softness may be formed two-dimensionally in the membrane (using phase separation phenomenon). These characteristics can be controlled by changing the temperature by externally applying electromagnetic stimuli or ultrasonic stimuli.
3. Liposomes, to which, other than the lipids, a charge-controlling agent, protein, and/or nonionic water-soluble polymer are optionally combined, are prepared. By this, a surface charge of the liposome membrane or molecule permeability is controlled at the same time as reducing tendencies thereof for deposition or aggregation, to thereby improve dispersion stability of the liposome.
4. Joining of the fullerenes and the liposomes is accelerated by using electrostatic attraction force of various ions, or adhesive force of protein to produce the liposome composition containing at least one liposome encapsulating or adsorbing at lest one fullerene therein, or thereon.
5. The liposome composition containing at least one liposome encapsulating or adsorbing at least one fullerene therein, or thereon is placed in a container filled with gas, and ultrasonic waves are applied thereto under the pressure to thereby make the gas included in the liposome composition.

In the manner mentioned above, the liposome composition of the present invention can be produced.

The liposome composition of the present invention is suitably used for medical uses.

For example, the liposome composition of the present invention can be used for treating various illnesses including cancer by using mechanical actions initiated by ultrasonic radiation, or active oxygen such as singlet oxygen and hydroxyl radicals generated by ultrasonic radiation.

The frequency of the ultrasonic wave for use in the radiation is suitably selected depending on the intended purpose without any restriction, but is preferably about 20 KHz to about 20 MHz, more preferably about 600 KHz to about 3 MHz.

The output of the radiation is suitably selected depending on the purpose without any restriction, but is preferably about 0.1 W/cm² to about 100 W/cm², more preferably about 0.5 W/cm² to about 10 W/cm².

The duty cycle of the ultrasonic wave is suitably selected depending on the intended purpose without any restriction, but is preferably about 1% to about 100%, more preferably about 10% to about 50%.

The duration of the ultrasonic radiation is suitably selected depending on the frequency, and output for use, without any restriction, but is preferably about 5 seconds to about 600 seconds, more preferably about 30 seconds to about 300 seconds.

The liposome composition of the present invention can be effectively used for treatments of various cancers, virus infections, intercellular parasite infections, pulmonary fibrosis, hepatic cirrhosis, chronic nephritis, arteriosclerosis, leukemia, and blood vessel stenosis.

Examples of the cancers include all solid cancers grown on the surface or inner part of organs, such as a lung cancer, liver cancer, pancreatic cancer, gastrointestinal cancer, bladder cancer, renal cancer, and brain tumor. Among them, the liposome composition of the present invention can be effectively used for a treatment of a cancer that is present in the deep part of a body, to which a photo-dynamic therapy cannot be performed.

With regard to other illness, as the focus or infected cell (affected cell) is located in the inner part of the organ, a treatment can be performed by accumulating the liposome composition of the present invention on such part using an appropriate method, and then externally applying ultrasonic waves.

### (Diagnostic Contrast Agent, Therapeutic Enhancer, Pharmaceutical Composition)

### <Diagnostic Contrast Agent>

The diagnostic contrast agent of the present invention contains at least the liposome composition of the invention, and may further contain other substances, if necessary.

The amount of the liposome composition contained in the diagnostic contrast agent is suitably selected depending on the intended purpose without any restriction. The diagnostic contrast agent may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the diagnostic contrast agent is suitably selected depending on the intended purpose without any restriction, provided that it does not adversely affect the obtainable effect of the liposome composition.

The diagnostic contrast agent may be used independently, or in combination with a medicine containing other substance(s) as an active ingredient. Moreover, the diagnostic contrast agent may be used by being formulated in a medicine containing other substance(s) as an active ingredient.

### <Therapeutic Enhancer>

The therapeutic enhancer of the present invention contains at least the liposome composition of the present invention, and may further contain other substances, if necessary.

The therapeutic enhancement is to exhibit a therapeutic effect of a therapeutic agent such as the liposome composition of the present invention, which has no or significantly small effect as a therapeutic effect when it is used singly, by applying physical energy such as ultrasonic wave, electronic field, or magnetic field, or to attain the increased therapeutic effect by combining physical energy such as ultrasonic waves, electronic field, or magnetic field, though the physical energy itself has no or significantly small therapeutic effect.

The amount of the liposome composition of the present invention contained in the therapeutic enhancer is suitably selected depending on the intended purpose without any restriction. The therapeutic enhancer may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the therapeutic enhancer is suitably selected depending on the intended purpose without any restriction, provided that it does not adversely affect the obtainable effect of the liposome composition.

The therapeutic enhancer may be used independently, or in combination with a medicine containing other substance(s) as an active ingredient. Moreover, the therapeutic enhancer may be used by being formulated in a medicine containing other substance(s) as an active ingredient.

### <Pharmaceutical Composition>

The pharmaceutical composition of the present invention contains at least the liposome composition of the present invention, and may further contain other substances, if necessary.

The amount of the liposome composition of the present invention contained in the pharmaceutical composition is suitably selected depending on the intended purpose without any restriction. The pharmaceutical composition may be the liposome composition of the present invention, itself.

Other substances are suitably selected, for example, from pharmacologically acceptable carriers, without any restriction. Examples thereof include ethanol, water, starch, saccharides, and dextran. The amount of other substances contained in the pharmaceutical composition is suitably selected depending on the intended purpose without any restriction, provided that it does not adversely affect the obtainable effect of the liposome composition.

The pharmaceutical composition may be used independently, or in combination with a medicine containing other substance(s) as an active ingredient. Moreover, the pharmaceutical composition may be used by being formulated in a medicine containing other substance(s) as an active ingredient.

### —Dosage Form—

The dosage form of the diagnostic contrast agent, therapeutic enhancer, and pharmaceutical composition is suitably selected depending on the intended purpose without any restriction. Examples thereof include parenteral injection (e.g., in vein, in artery, in muscle, subcutis, and intracutaneous), a dispersing agent, and liquids. The diagnostic contrast agent, therapeutic enhancer, and pharmaceutical composition of these dosage forms can be produced in accordance with the conventional methods. In the case where it is administered as parenteral injection, for example, parenteral injection can be attained by formulating the liposome composition of the present invention with various additives generally used for parenterial injection, such as buffer, physiological saline, preservatives, distilled water for injection and the like.

### —Administration—

The administration method of the diagnostic contrast agent, therapeutic enhancer, and pharmaceutical composition is suitably selected depending on the dosage form thereof without any restriction.

The dosage of the diagnostic contrast agent, therapeutic enhancer, and pharmaceutical composition is suitably selected, without any restriction, considering various factors, such as administrating path, age and sex of a patient, and a type and situation of illness. For example, in the case of an adult, it can be administered in an amount of about 0.01 mg/kg to about 10 mg/kg per day, which will be taken at once or separately in a few times.

The period for administering the diagnostic contrast agent, therapeutic enhancer, and pharmaceutical composition is suitably selected depending on the intended purpose without any restriction.

The animal species to be a subject of an administration of the diagnostic contrast agent, therapeutic enhancer, and pharmaceutical composition are suitably selected depending on the intended purpose without any restriction. Examples thereof include humans, monkeys, pigs, cattle, sheep, goats, dogs, cats, mice, rats, and birds.

The liposome composition of the present invention is excellent in dispersion stability in an aqueous solvent in a neutral pH range, and has high diagnostic and therapeutic effect in assistance with ultrasonic waves, as it includes at least one liposome entrapping gas therein, and encapsulating or adsorbing fullerene(s) therein or thereon. Moreover, since the liposome composition of the present invention can accurately visualize the distribution of the gas by a ultrasonic diagnostic equipment, a treatment can be carried out at the same time as highly accurately detecting a lesioned part such as cancer. Therefore, the liposome composition of the present invention contributes to a quality of life (QOL) of a patient.

### EXAMPLES

The present invention will be more specifically explained with Examples hereinafter, but these Examples shall not be construed as limiting the scope of the present invention. Moreover, any modification, which is made in Examples so as not to depart from the meaning of the prior or posterior description, will be included in the technical scope of the present invention.

### Comparative Example 1-1

Fullerenes C₇₀ (100 mg), γ-cyclodextrin (700 mg), and zirconium beads each having a diameter of 1 mm (20 g) were mixed, and dispersed by means of a Planetary Ball Mill PM100 (manufactured by Retsch Co., Ltd.) for 30 minutes. To this dispersion, 40 mL of water was added, to thereby obtain an aqueous dispersion of C₇₀.

To COATSOME EL-01-N (containing 54 μl of L-α-dipalmitoylphosphatidylcholine (DPPC), 40 μl of cholesterol (CHOL), and 6 μmol of L-α-dipalmitoylphosphatidylglycerol) manufactured by NOF CORPORATION, the aforementioned aqueous dispersion of C₇₀ was added in an amount of 2 mL after passing the aqueous dispersion through a filter having a pore diameter of 0.2 μm and then the mixture was vibrated to thereby prepare a weakly negatively-charged liposome composition dispersion liquid (C₇₀ content of 0.34 mg/mL), that was a liposome composition dispersion liquid of Comparative Example 1-1 (hereinafter, may be referred to as Sample 1A).

A volume average dispersed-particle diameter of Sample 1A was measured by means of a microtrack UPA-UT151 particle size analyzer (manufactured by Nikkiso Co., Ltd.), and it was 210 nm.

Sample 1A was stable in a PBS buffer solution (pH 7.2) (under physiological conditions). Note that, the “stable” means the state where no aggregation or precipitation occurs therein after it was left to stand under physiological conditions at 25° C. for 24 hours.

### Example 1-1

Sample 1A obtained in Comparative Example 1-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 1-1 (hereinafter, may be referred to as Sample 1B).

A volume average dispersed-particle diameter of Sample 1B was measured in the same manner as in Comparative Example 1-1, and it was 280 nm.

A concentration of the perfluoropropane gas in Sample 1B was determined by a gas chromatograph GC-2014 (manufactured by Shimadzu Corporation), and it was 2.8 μL/mL.

Sample 1B was stable in a PBS buffer solution (pH 7.2).

### Comparative Example 1-2

To COATSOME EL-01-N manufactured by NOF CORPORATION, 2 mL of pure water was added, and the mixture was vibrated to prepare a weakly negatively-charged liposome dispersion liquid (hereinafter, referred to as Sample 1C), that was a liposome dispersion liquid of Comparative Example 1-2 (hereinafter, may be referred to as Sample 1C).

A volume average dispersed-particle diameter of Sample 1C was measured in the same manner as in Comparative Example 1-1, and it was 270 nm.

Sample 1C was stable in a PBS buffer solution (pH 7.2).

### Comparative Example 1-3

Sample 1C obtained in Comparative Example 1-2 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Comparative Example 1-3 (hereinafter, may be referred to as Sample 1D).

A volume average dispersed-particle diameter of Sample 1D was measured in the same manner as in Comparative Example 1-1, and it was 300 nm.

A concentration of the perfluoropropane gas in Sample 1D was determined in the same manner as in Example 1-1, and it was 2.6 μL/mL.

Sample 1D was stable in a PBS buffer solution (pH 7.2).

### Comparative Example 2-1

A liposome composition dispersion liquid of Comparative Example 2-1 (hereinafter, may be referred to as Sample 2A) was prepared in the same manner as in Comparative Example 1-1, provided that COATSOME EL-01-N was replaced with a mixture of 1,2-distearoyl-sn-glycero-phosphatidylcholine (DSPC) (94 μmol) and 1,2-distearoyl-sn-glycero-3-phosphatidyl-ethanolamine-methoxy-polyethylene glycol (DSPE-PEG) (6 μmol) to form liposomes.

A volume average dispersed-particle diameter of Sample 2A was measured in the same manner as in Comparative Example 1-1, and it was 340 nm.

Sample 2A was stable in a PBS buffer solution (pH 7.2).

### Example 2-1

Sample 2A obtained in Comparative Example 2-1 was poured into a vial, and the vial was filled with perfluoropropane (PFP) gas. After filling the vial with the gas in the volume that was 1.5 times of the volume of the vial under pressure, ultrasonic waves of 20 kHz and 50 W were applied thereto for 15 minutes. Thereafter, ultrasonic waves of 800 kHz and 30 W were further applied for 60 minutes to thereby obtain a liposome composition dispersion liquid of Example 2-1 (hereinafter, may be referred to as Sample 2B).

A volume average dispersed-particle diameter of Sample 2B was measured in the same manner as in Comparative Example 1-1, and it was 370 nm.

A concentration of the perfluoropropane gas in Sample 2B was determined in the same manner as in Example 1-1, and it was 2.5 μL/mL.

Sample 2B was stable in a PBS buffer solution (pH 7.2).

### Example 2-2

A liposome composition dispersion liquid of Example 2-2 (hereinafter, may be referred to as Sample 2C) was prepared in the same manner as in Example 2-1, provided that the perfluoropropane (PFP) gas was replaced with air.

A volume average dispersed-particle diameter of Sample 2C was measured in the same manner as in Comparative Example 1-1, and it was 390 nm.

A concentration of the air in Sample 2C was determined in the same manner as in Example 1-1, and it was 2.4 μL/mL.

Sample 2C was stable in a PBS buffer solution (pH 7.2).

### Example 2-3

A liposome composition dispersion liquid of Example 2-3 (hereinafter, may be referred to as Sample 2D) was prepared in the same manner as in Example 2-1, provided that the perfluoropropane (PFP) gas was replaced with xenon (Xe) gas.

A volume average dispersed-particle diameter of Sample 2D was measured in the same manner as in Comparative Example 1-1, and it was 370 nm.

A concentration of the xenon gas in Sample 2D was determined in the same manner as in Example 1-1, and it was 2.8 μL/mL.

Sample 2D was stable in a PBS buffer solution (pH 7.2).

### Example 2-4

A liposome composition dispersion liquid of Example 2-4 (hereinafter, may be referred to as Sample 2E) was prepared in the same manner as in Example 2-1, provided that the perfluoropropane (PFP) gas was replaced with krypton (Kr) gas.

A volume average dispersed-particle diameter of Sample 2E was measured in the same manner as in Comparative Example 1-1, and it was 360 nm.

A concentration of the krypton gas in Sample 2E was determined in the same manner as in Example 1-1, and it was 2.7 μL/mL.

Sample 2E was stable in a PBS buffer solution (pH 7.2).

### Example 2-5

A liposome composition dispersion liquid of Example 2-5 (hereinafter, may be referred to as Sample 2F) was prepared in the same manner as in Example 2-1, provided that the perfluoropropane (PFP) gas was replaced with argon (Ar) gas.

A volume average dispersed-particle diameter of Sample 2F was measured in the same manner as in Comparative Example 1-1, and it was 370 nm.

A concentration of the argon gas in Sample 2F was determined in the same manner as in Example 1-1, and it was 2.6 μL/mL.

Sample 2F was stable in a PBS buffer solution (pH 7.2).

### Example 2-6

A liposome composition dispersion liquid of Example 2-6 (hereinafter, may be referred to as Sample 2G) was prepared in the same manner as in Example 2-1, provided that the perfluoropropane (PFP) gas was replaced with 1,1,1,2,3,4,4,5,5,5-decafluoropentane.

A volume average dispersed-particle diameter of Sample 2G was measured in the same manner as in Comparative Example 1-1, and it was 360 nm.

A concentration of 1,1,1,2,3,4,4,5,5,5-decafluoropentane in Sample 2G was determined in the same manner as in Example 1-1, and it was 2.4 μL/mL.

Sample 2G was stable in a PBS buffer solution (pH 7.2).

### Comparative Example 3-1

A liposome composition dispersion liquid of Comparative Example 3-1 (hereinafter, may be referred to as Sample 3A) was prepared in the same manner as in Comparative Example 1-1, provided that the fullerenes C₇₀ was replaced with fullerenes C₆₀.

A volume average dispersed-particle diameter of Sample 3A was measured in the same manner as in Comparative Example 1-1, and it was 220 nm.

Sample 3A was stable in a PBS buffer solution (pH 7.2).

### Example 3-1

A liposome composition dispersion liquid of Example 3-1 (may be referred to as Sample 3B) was prepared in the same manner as in Example 1-1, provided that Sample 1A was replaced with Sample 3A.

A volume average dispersed-particle diameter of Sample 3B was measured in the same manner as in Comparative Example 1-1, and it was 260 nm.

A concentration of the perfluoropropane gas in Sample 3B was determined in the same manner as in Example 1-1, and it was 2.9 μL/mL.

Sample 3B was stable in a PBS buffer solution (pH 7.2).

### Comparative Example 4-1

Aqueous dispersion liquid of water-soluble hydroxylated fullerenes (C₆₀)2 was prepared in accordance with the description of Example 1, JP-A No. 2007-176899. The amount of the water-soluble hydroxylated fullerenes 2 in the aqueous dispersion liquid was 2 mg/mL.

In the same manner as in Comparative Example 1-1, 2 mL of the water-soluble hydroxylated fullerenes 2 dispersion liquid was added to COATSOME EL-01-N, and the mixture was vibrated to prepare a weakly negatively-charged liposome composition dispersion liquid (the water-soluble hydroxylated fullerene 2 content: 0.52 mg/mL), that was a liposome dispersion liquid of Comparative Example 4-1 (hereinafter, may be referred to as Sample 4A).

A volume average dispersed-particle diameter of Sample 4A was measured in the same manner as in Comparative Example 1-1, and it was 220 nm.

Sample 4A was stable in a PBS buffer solution (pH 7.2).

### Example 4-1

A liposome composition dispersion liquid of Example 4-1 (hereinafter, may be referred to as Sample 4B) was prepared in the same manner as in Example 1-1, provided that Sample 1A was replaced with Sample 4A.

A volume average dispersed-particle diameter of Sample 4B was measured in the same manner as in Comparative Example 1-1, and it was 290 nm.

A concentration of the perfluoropropane gas in Sample 4B was determined in the same manner as in Example 1-1, and it was 2.5 μL/mL.

Sample 4B was stable in a PBS buffer solution (pH 7.2).

### Comparative Example 5-1

Fullerenes C₇₀ were made encapsulated in DTP-DOPE-containing PEG-modified liposomes in the manner described in Example 1, JP-A No. 2005-298486, provided that 6.0 mL of 250 mM ammonium sulfate solution was replaced with 6.0 mL of Sample 1A (0.034% by mass C₇₀ dispersion liquid). Note that, DTP, DOPE, and PEG mentioned above are 3-(2-pyridyldithio)propionitrile, 1,2-dioleyl-sn-glycero-3-phosphoethanol amine, and polyethylene glycol, respectively.

The DTP-DOPE containing PEG-modified liposome composition containing C₇₀ was then bonded to rHSA (genetically-modified human serum albumin) in the manner described in Example 1, JP-A No. 2005-298486 to obtain C₇₀-containing PEG-rHSA-modified liposome composition of Comparative Example 5-1 (hereinafter, may be referred to as Sample 5A).

A volume average dispersed-particle diameter of Sample 5A was measured in the same manner as in Comparative Example 1-1, and it was 170 nm.

Sample 5A was stable in a PBS buffer solution (pH 7.2).

### Example 5-1

A liposome composition dispersion liquid of Example 5-1 (hereinafter, may be referred to as Sample 5B) was prepared in the same manner as in Example 1-1, provided that Sample 1A was replaced with Sample 5A.

A volume average dispersed-particle diameter of Sample 5B was measured in the same manner as in Comparative Example 1-1, and it was 210 nm.

A concentration of the perfluoropropane gas in Sample 5B was determined in the same manner as in Example 1-1, and it was 2.5 μL/mL.

Sample 5B was stable in a PBS buffer solution (pH 7.2).

The constitutions of liposome compositions obtained in Examples 1-1 to 5-1, and Comparative Examples 1-1 to 5-1 are summarized in Table 1.

TABLE 1

| | | | | | | | Liposome | |
|---|---|---|---|---|---|---|---|---|
| | | | | | | | | Volume |
| | | | | | | | | average |
| | | | | | | | | dispersed |
| | | Fullerene | | Gas | | | | particle |
| | | | mass | | content | concentration | | diameter |
| | Sample | type | (mg) | type | (μL) | (μL/mL) | type | (nm) |
| Comp. | 1A | C₇₀ | 0.68 | — | — | — | COATSOME | 210 |
| Ex. | | | | | | | EL-01-N | |
| 1-1 | | | | | | | | |
| Ex. | 1B | C₇₀ | 0.68 | PFP | 5.6 | 2.8 | COATSOME | 280 |
| 1-1 | | | | | | | EL-01-N | |
| Comp. | 1C | — | — | — | — | — | COATSOME | 270 |
| Ex. | | | | | | | EL-01-N | |
| 1-2 | | | | | | | | |
| Comp. | 1D | — | — | PFP | 5.2 | 2.6 | COATSOME | 300 |
| Ex. | | | | | | | EL-01-N | |
| 1-3 | | | | | | | | |
| Comp. | 2A | C₇₀ | 0.68 | — | — | — | DSPC + | 340 |
| Ex. | | | | | | | DSPE-PEG | |
| 2-1 | | | | | | | | |
| Ex. | 2B | C₇₀ | 0.68 | PFP | 5 | 2.5 | DSPC + | 370 |
| 2-1 | | | | | | | DSPE-PEG | |
| Ex. | 2C | C₇₀ | 0.68 | air | 4.8 | 2.4 | DSPC + | 390 |
| 2-2 | | | | | | | DSPE-PEG | |
| Ex. | 2D | C₇₀ | 0.68 | xenon | 5.6 | 2.8 | DSPC + | 370 |
| 2-3 | | | | | | | DSPE-PEG | |
| Ex. | 2E | C₇₀ | 0.68 | krypton | 5.4 | 2.7 | DSPC + | 360 |
| 2-4 | | | | | | | DSPE-PEG | |
| Ex. | 2F | C₇₀ | 0.68 | argon | 5.2 | 2.6 | DSPC + | 370 |
| 2-5 | | | | | | | DSPE-PEG | |
| Ex. | 2G | C₇₀ | 0.68 | decafluoro | 4.8 | 2.4 | DSPC + | 360 |
| 2-6 | | | | pentane | | | DSPE-PEG | |
| Comp. | 3A | C₆₀ | 0.6 | — | — | — | COATSOME | 220 |
| Ex. | | | | | | | EL-01-N | |
| 3-1 | | | | | | | | |
| Ex. | 3B | C₆₀ | 0.6 | PFP | 5.8 | 2.9 | COATSOME | 260 |
| 3-1 | | | | | | | EL-01-N | |
| Comp. | 4A | water-soluble | 1.04 | — | — | — | COATSOME | 220 |
| Ex. | | hydrogenated | | | | | EL-01-N | |
| 4-1 | | fullerene | | | | | | |
| Ex. | 4B | water-soluble | 1.04 | PFP | 5 | 2.5 | COATSOME | 290 |
| 4-1 | | hydrogenated | | | | | EL-01-N | |
| | | fullerene | | | | | | |
| Comp. | 5A | C₇₀ | 0.71 | — | — | — | PEG•rHSA | 170 |
| Ex. | | | | | | | modified | |
| 5-1 | | | | | | | liposome | |
| Ex. | 5B | C₇₀ | 0.71 | PFP | 5 | 2.5 | PEG•rHSA | 210 |
| 5-1 | | | | | | | modified | |
| | | | | | | | liposome | |

### Experimental Example 1

### Cancer Cell Killing Test I by Ultrasonic Radiation

Using a human lymphoma cell strain U937, cell-killing effect of each sample was examined by applying ultrasonic wave to each sample.

RPMI 1640 to which 10% FBS had been added was used as a culture solution, and a concentration of cells was adjusted to 1×10⁶ cells/mL. In a 96-well cell culture plate, a cell suspension and the aforementioned sample were both added in an amount of 180 μL and 20 μL, respectively, per well. To this, ultrasonic waves were applied at the intensity of 0.5 W/cm², duty rate of 50% by means of a sonoporator SP-100 (Sonidel Limited) for 10 seconds. After the application of ultrasonic waves, the mixture of the cells and sample was incubated by an incubator in the atmosphere of CO₂ at 37° C. for 2 hours. Thereafter, a number of living cells was determined and evaluated by a trypan blue-exclusion test. The results are shown in Table 2.

TABLE 2

| | | Number of |
|---|---|---|
| | Sample | living cells |
| Comp. Ex. 1-1 | 1A | 68 |
| Ex. 1-1 | 1B | 41 |
| Comp. Ex. 1-2 | 1C | 102 |
| Comp. Ex. 1-3 | 1D | 79 |
| Comp. Ex. 2-1 | 2A | 71 |
| Ex. 2-1 | 2B | 39 |
| Ex. 2-2 | 2C | 50 |
| Ex. 2-3 | 2D | 46 |
| Ex. 2-4 | 2E | 49 |
| Ex. 2-5 | 2F | 51 |
| Ex. 2-6 | 2G | 45 |
| Comp. Ex. 3-1 | 3A | 89 |
| Ex. 3-1 | 3B | 45 |
| Comp. Ex. 4-1 | 4A | 90 |
| Ex. 4-1 | 4B | 51 |
| Comp. Ex. 5-1 | 5A | 73 |
| Ex. 5-1 | 5B | 43 |

From the results shown in Table 2, it can be seen that the liposome composition of the present invention in which the liposome entraps the gas, and encapsulates or adsorbs fullerenes had excellent cancer cell killing effects.

### Experimental Example 2

### Cancer Cell Killing Test II by Ultrasonic Radiation

Experimental Example 2 was carried out in the same manner as in Experimental Example 1, provided that the human lymphoma cell strain U937 was replaced with a human cervical cancer cell strain (Hela cells), and the culture solution was changed from RPMI 1640 to which 10% FBS had been added to MEN to which 10% FBS and 1% NEAA had been added. The results are shown in Table 3.

TABLE 3

| | | Number of |
|---|---|---|
| | Sample | living cells |
| Comp. Ex. 1-1 | 1A | 78 |
| Ex. 1-1 | 1B | 47 |
| Comp. Ex. 1-2 | 1C | 129 |
| Comp. Ex. 1-3 | 1D | 91 |
| Comp. Ex. 2-1 | 2A | 80 |
| Ex. 2-1 | 2B | 43 |
| Ex. 2-2 | 2C | 69 |
| Ex. 2-3 | 2D | 52 |
| Ex. 2-4 | 2E | 57 |
| Ex. 2-5 | 2F | 60 |
| Ex. 2-6 | 2G | 50 |
| Comp. Ex. 3-1 | 3A | 77 |
| Ex. 3-1 | 3B | 48 |
| Comp. Ex. 4-1 | 4A | 80 |
| Ex. 4-1 | 4B | 53 |
| Comp. Ex. 5-1 | 5A | 82 |
| Ex. 5-1 | 5B | 51 |

From the results shown in Table 3, it can be seen that the liposome composition of the present invention in which the liposome entraps the gas, and encapsulates or adsorbs fullerenes had excellent cancer cell killing effects.

### Example 6-1

In the course of preparing the commercial ultrasonic diagnostic contrast agent, SONAZOID for Injection 16 μL (manufactured by Daiichi Sankyo Company, Limited), instead of using the attached water for injection (2 mL), liquid which was prepared by passing the C₇₀ aqueous dispersion liquid, which had been prepared in Comparative Example 1-1, through a filter having a pore diameter of 0.2 μm was added, and the mixture was vibrated to prepare a liposome dispersion liquid (C₇₀ content: 0.34 mg/mL), to thereby obtain a liposome composition dispersion liquid of Example 6-1 (hereinafter, may be referred to as Sample 6A).

A volume average dispersed-particle diameter of Sample 6A was measured in the same manner as in Comparative Example 1-1, and it was 3.5 μm.

A concentration of the perfluoropropane gas in Sample 6A was determined in the same manner as in Example 1-1, and it was 7.9 μL/mL.

Sample 6A was stable in a PBS buffer solution (pH 7.2).

### Example 6-2

A liposome composition dispersion liquid of Example 6-2 (hereinafter, may be referred to as Sample 6B) was prepared in the same manner as in Example 6-1, provided that the C₇₀ aqueous dispersion liquid was replaced with the water-soluble hydroxylated fullerene (C₆₀) 2 prepared in Comparative Example 4-1 to prepare a liposome dispersion liquid (hydroxylated fullerene content: 0.52 mg/mL).

A volume average dispersed-particle diameter of Sample 6B was measured in the same manner as in Comparative Example 1-1, and it was 3.4 μm.

A concentration of the perfluoropropane gas in Sample 6B was determined in the same manner as in Example 1-1, and it was 7.9 μL/mL.

Sample 6B was stable in a PBS buffer solution (pH 7.2).

### Example 6-3

A liposome composition dispersion liquid of Example 6-3 (hereinafter, may be referred to as Sample 6C) was prepared in the same manner as in Example 6-2, provided that the formulated amount of the water-soluble hydroxylated fullerene (C₆₀) 2 was changed to 1.90 mg/mL.

A volume average dispersed-particle diameter of Sample 6C was measured in the same manner as in Comparative Example 1-1, and it was 3.2 μm.

A concentration of the perfluoropropane gas in Sample 6C was determined in the same manner as in Example 1-1, and it was 7.8 μL/mL.

Sample 6C was stable in a PBS buffer solution (pH 7.2).

### Example 6-4

A liposome composition dispersion liquid of Example 6-4 (hereinafter, may be referred to as Sample 6D) was prepared in the same manner as in Example 6-2, provided that the formulated amount of the water-soluble hydroxylated fullerene (C₆₀) 2 was changed to 0.03 mg/mL.

A volume average dispersed-particle diameter of Sample 6D was measured in the same manner as in Comparative Example 1-1, and it was 3.8 μm.

A concentration of the perfluoropropane gas in Sample 6D was determined in the same manner as in Example 1-1, and it was 8.0 μL/mL.

Sample 6D was stable in a PBS buffer solution (pH 7.2).

The constitutions of liposomes obtained in Examples 6-1 to 6-4 are summarized in Table 4.

TABLE 4

| | | | | | | | Liposome composition | | |
|---|---|---|---|---|---|---|---|---|---|
| | | Fullerene | | Gas | | | | volume average | stability under |
| | | | mass | | content | concentration | | dispersed particle | physiological |
| | Sample | type | (mg) | type | (μL) | (μL/mL) | type | diameter (nm) | conditions |
| Commercial | SONAZOID | — | — | perfluoro- | 16.0 | 8.0 | hydrogenated egg | 3.8 | stable |
| product | | | | butane | | | phosphatidyl | | |
| | | | | | | | serine sodium salt | | |
| Ex. | 6A | C₇₀ | 0.68 | perfluoro- | 15.8 | 7.9 | hydrogenated egg | 3.5 | stable |
| 6-1 | | | | butane | | | phosphatidyl | | |
| | | | | | | | serine sodium salt | | |
| Ex. | 6B | water-soluble | 1.04 | perfluoro- | 15.8 | 7.9 | hydrogenated egg | 3.4 | stable |
| 6-2 | | hydroxylated | | butane | | | phosphatidyl | | |
| | | fullerene | | | | | serine sodium salt | | |
| Ex. | 6C | water-soluble | 3.80 | perfluoro- | 15.6 | 7.8 | hydrogenated egg | 3.2 | stable |
| 6-3 | | hydroxylated | | butane | | | phosphatidyl | | |
| | | fullerene | | | | | serine sodium salt | | |
| Ex. | 6D | water-soluble | 0.06 | perfluoro- | 16.0 | 8.0 | hydrogenated egg | 3.8 | stable |
| 6-4 | | hydroxylated | | butane | | | phosphatidyl | | |
| | | fullerene | | | | | serine sodium salt | | |

### Experimental Example 3

### Growth Inhibition Test of Melanoma on Mice by Ultrasonic Radiation

Female nude mice of 5 weeks old were used for the test, and 100 μL of melanoma cells (C32 cells) adjusted to 2×10⁷ cell (cell viability ≧98%) was hypodermically injected to each mouse. When the tumor was grown to have the diameter of approximately 5 mm, a treatment was started. For a treatment, the mice were randomly separated into 6 groups (5 mice in each group), for six different treatments including a ultrasonic treatment only, SONAZOID with a ultrasonic treatment, and each of Samples 6A to 6D with a ultrasonic treatment. While giving the mice inhalation anesthesia, 10 μL of the sample was locally injected to the mice of each group, and ultrasonic waves were applied thereto at a frequency of 1 MHz, intensity of 1 W/cm², and duty ratio of 50% for 2 minutes by means of a sonoporation SONITRON 1000 (manufactured by Rich-Mar Corp.). For comparison, 5 mice whose tumors were not treated were also provided.

The injection of the sample and ultrasonic radiation were both performed every other day, 5 times in total, and the size of the tumor (represented as a product of the long axis and the short axis) was measured in two weeks after the last treatment. The results are shown in Table 5.

TABLE 5

| | | Size of tumor (cm²) |
|---|---|---|
| | Sample | (average of 5 mice) |
| Commercial | SONAZOID | 123 |
| product | | |
| Ex. 6-1 | 6A | 72 |
| Ex. 6-2 | 6B | 69 |
| Ex. 6-3 | 6C | 61 |
| Ex. 6-4 | 6D | 102 |
| Ultrasonic | — | 128 |
| only | | |
| No | — | 150 |
| treatment | | |

From the results shown in Tables 4 and 5, it can be seen that the liposome composition of the present invention, in which the liposome entraps the gas therein, and encapsulates or adsorbs fullerenes therein or thereon, is stably dispersed under physiological conditions, and the liposome composition of the present invention exhibits an effect of inhibiting the growth of melanoma on mice so that it is effective as a therapeutic enhancer.

### Experimental Example 4

### Liver Cancer Cystography Test on Rats by Ultrasonic Radiation

Cancer cells were implanted to rats in advance, and 10 μL of each of SONAZOID and Samples 6A to 6D was injected to a tail vein of each rat. After a certain period, an ultrasonography was performed by a harmonic method (TOSHIBA Ultrasound Aplio 80 (manufactured by Toshiba Medical Systems Corporation)). As a result, all the samples provided the same degree of accuracy and contrast in the obtained image of the liver cancer to that with SONAZOID.

Accordingly, it was found that the liposome composition of the present invention in which the liposome entraps the air thereof, and encapsulates or adsorbs fullerenes therein or thereon was effective as a diagnostic contrast agent.

The liposome composition of the present invention in which the liposome entraps the gas therein, and encapsulate or adsorbs fullerenes therein or thereon has excellent dispersion stability in an aqueous medium in the neutral pH range, and is suitably used, for example, as a diagnostic contrast agent, therapeutic enhancer, and pharmaceutical composition, which are used for diagnoses and therapies mainly using ultrasonic waves.

Moreover, since the liposome composition of the present invention can accurately visualize the distribution of the gas by a ultrasonic diagnostic equipment, a treatment can be carried out at the same time as highly accurately detecting a lesioned part such as cancer. Therefore, the liposome composition of the present invention contributes to a quality of life (QOL) of a patient.

What is claimed is:

1. A liposome composition, comprising:
at least one liposome;
gas entrapped in the liposome; and
at least one fullerene encapsulated in or adsorbed on the liposome.

2. The liposome composition according to claim 1, wherein the liposome composition has a volume average dispersed-particle diameter of 20 nm to 20 μm.

3. The liposome composition according to claim 1, wherein the gas is at least one selected from the group consisting of oxygen, nitrogen, carbon dioxide, xenon, krypton, argon, hydrofluorocarbons, and perfluorocarbons.

4. The liposome composition according to claim 1, wherein the fullerene is at least one selected from the group consisting of C₆₀, C₇₀, and derivatives thereof.

5. The liposome composition according to claim 1, further comprising a receptor bonded to or contained in the liposome, wherein the receptor is capable of specifically recognizing a certain tissue.

6. The liposome composition according to claim 1, wherein the liposome composition is ultrasonic sensitive.

7. The liposome composition according to claim 1, wherein the liposome composition is used for a medical use.

8. A diagnostic contrast agent, comprising:
a liposome composition,
wherein the liposome composition comprises at least one liposome, gas entrapped in the liposome, and at least one fullerene encapsulated in or adsorbed on the liposome.

9. A therapeutic enhancer, comprising:
a liposome composition,
wherein the liposome composition comprises at least one liposome, gas entrapped in the liposome, and at least one fullerene encapsulated in or adsorbed on the liposome.

10. A pharmaceutical composition, comprising:
a liposome composition,
wherein the liposome composition comprises, at least one liposome, gas entrapped in the liposome, and at least one fullerene encapsulated in or adsorbed on the liposome.
